(19) United States
(12) Patent Application Publication
(10) Pub. No.: US 2009/0130212 A1
(43) Pub. Date: May 21, 2009

(54) COMPOSITION AND IMPROVED METHOD FOR PREPARATION OF SMALL PARTICLES

(75) Inventor: Robert A. Bellantone, Yonkers, NY (US)

Correspondence Address: HOFFMANN & BARON, LLP, 6900 JERICHO TURNPIKE, SYOSSET, NY, 11791, US

(73) Assignee: PHYSICAL PHARMACEUTICA, LLC, Yonkers, NY (US)

(21) Appl. No.: 12/300,659

(22) PCT Filed: May 15, 2007
(86) PCT No.: PCT/US2007/011591

§ 371 Date: Nov. 13, 2008

Related U.S. Application Data
(60) Provisional application No. 60/800,462, filed on May 15, 2006.

(51) Int. Cl. A61K 9/10; A61K 31/192

(52) U.S. Cl. 424/484; 514/570

(57) ABSTRACT

The present invention relates to a novel method of loading drug molecules into small pores, along with the composition so produced. In a preferred embodiment, the drug is dissolved in a suitable solvent (which may or may not be biocompatible), and the solution is allowed to move into the pores of solid matrixes by, e.g., capillary action, optionally under the influence of pressure or vacuum. The drug is then precipitated in the pores by evaporating the solvent faster than the drug can diffuse out of the pores, which leaves solid drug particles that are not larger than the pore. Since the pore radii in solid pharmaceutical matrixes can be as small as several nanometers, the drug particle size range includes particles that are much smaller than those produced using current methods. The solvent may be a pure material, a combination of solvents, a combination of liquids and surfactants, or a supercritical fluid with or without surfactants.

### BACKGROUND OF THE INVENTION

1. Field of the Invention

The invention relates to an improved method of producing small particles of materials, especially those having poor solubility in water, e.g., solid drug particles, that can increase aqueous solubility of the materials and thus the bioavailability of poorly soluble drugs. In particular, the invention relates to the use of an improved method of making drug particles with a small particle size, preferably nanoparticles, by depositing, e.g., by precipitating the drug in the pores or other voids of another, preferably solid material, which may or may not have significantly greater aqueous solubility than that of the drug. The size of a drug particle should not exceed the size of the pore in which it resides, and thus can be characterized by measuring the distribution of pore sizes in the matrix before and after the drug deposition. In addition, the practical performance of these drug forms can be characterized. Examples of such characterization include measuring the increase in drug solubility and/or dissolution rates in vitro, and enhanced absorption of drugs from the GI tract in vivo. (Although these examples involve drugs and are of pharmaceutical interest, the scope of this invention extends to include the particle formation, solubility and/or dissolution of any suitable material.)

2. Summary of the Prior Art

Typically, when drugs or other solutes are precipitated from a solution, there is no preferred or natural particle size for the resulting solid particles, and a wide range of particle sizes results. The particle size range depends on a number of factors, such as the solute concentration, rate of nucleation and crystallization, etc. Thus, it is difficult to predict the particle size. For most pharmaceuticals, precipitation due to lowering the temperature or evaporating the solvent produces particles in the micron range (typically 10-100 microns). While it is possible that there might be some particles in the distribution that would be smaller than this range, only a small percentage of these particles would be submicron in size, and a negligible fraction would be less than 0.1-0.2 micron in size.

Particle size reduction to nanometer scale is a known method for increasing the aqueous solubility and bioavailability of poorly water soluble materials, particularly drugs. However, certain deficiencies, as will be discussed more fully below, have prevented its optimum application. The prior art technology is based on two methods-mechanical processes (in which larger particles are mechanically milled or broken down to smaller ones) and precipitation-based processes. A useful survey of prior art techniques is given in Kharb et al, “Nanoparticle Technology for the Delivery of Poorly Water-Soluble Drugs,” Pharmaceutical Technology, Feb. 2, 2006.

Mechanical processes include wet milling and high pressure homogenization. Wet milling is a process in which drug crystals are dispersed in an aqueous surfactant solution, and mechanically ground (milled) in the presence of a grinding medium, such as glass or zirconium oxide particles that are typically 0.5-3 microns (500-3,000 nm) in diameter. High pressure homogenization is done by suspending drug crystals in an aqueous surfactant solution and using high-pressure (1100-1500 atmosphere) air jets to force the suspension through small gaps repeatedly, which results in the drug particles being mechanically broken up into smaller pieces. Wet milling suffers from the possibility of particle contamination from the grinding medium, which is avoided in high pressure homogenization. However, high pressure homogenization can sometimes change the crystal structure of the drug and create amorphous regions. While the presence of amorphous regions can increase the solubility of a drug, it can also be a significant drawback because of possible drug instability and quality control problems.

Emulsification is an example of a precipitation method. In this method, the drug is dissolved in an organic solvent, which is then dispersed in an aqueous surfactant solution to form an emulsion with an aqueous external phase. The organic solvent is then removed, and the drug precipitates into a suspension of small particles surrounded by surfactant molecules. The removal of organic solvent can be done by evaporation at a reduced pressure, or by diluting the external phase enough for the organic phase to diffuse away, leaving a suspension of nanoparticles in water.

Another precipitation process is precipitation with a compressed fluid antisolvent (PCA). There, supercritical carbon dioxide is mixed with organic solvents containing drug compounds. (A supercritical material can be either liquid or gas, and is used above the critical temperature and pressure where gases and liquids coexist.). The organic solvents expand into the carbon dioxide, leaving less solvent to dissolve the drug. As a result, the drug solution becomes supersaturated, which leads to precipitation of the drug.

Another precipitation process is rapid expansion from a liquefied-gas solution (RESS). In an RESS process, a solution or dispersion of surfactant in the supercritical fluid is formed, and rapid nucleation is induced. However, since the surfactant inhibits the particle growth, the result is the formation of more particles that are smaller. RESS can also be combined with high-pressure homogenization.

Spray freezing into liquid (SFL) is a process in which a drug is dissolved in a solvent (aqueous, organic, or aqueous plus an organic cosolvent), or dispersed in an emulsion. Subsequently, the liquid (solution or dispersion) is sprayed or atomized into liquid nitrogen to produce frozen particles containing the drug. The final nanoparticles are produced by lyophilizing the solvents to obtain a powder. Nanoparticles produced by this method are often amorphous.

Another method is known as evaporative precipitation into aqueous solution (EPAS). In this process, a drug is dissolved in an organic solvent with a low boiling point, and the solution is heated under pressure to a temperature above the normal boiling point of the solvent. The solution is sprayed or atomized into an aqueous surfactant solution that is also at a temperature above the organic solvent boiling point. The organic solvent then disperses into the hot water and abandons the drug, leaving a drug/surfactant solution which precipitates before or during cooling.

Other methods have claimed to produce nanoparticles, but these are not particles of the pure drug. For instance, it has been reported that ibuprofen can be complexed with cyclodextrin. However, this is a molecular effect. Another method has been to coat drug particles and biological agents, such as DNA and proteins, with compounds such as PLGA (polylactic-co-glycolic acid). However, these particles are typically close to a micron in size, or larger.

It will be apparent from the above discussion that the processes of the prior art are cumbersome, complex and expensive, either chemically or mechanically or both. In contrast, the present invention provides a simpler, better controlled and more economical method for producing small particles, especially nanoparticles, and the compositions produced thereby.

### BRIEF DESCRIPTION OF THE DRAWINGS

FIG. 1 shows a schematic diagram of an empty pore in a solid matrix particle.

FIG. 2 shows a schematic diagram of a pore in a solid matrix particle that is filled with a solution containing a drug.

FIG. 3 shows a schematic diagram of a pore in a solid matrix particle that contains particles of solid drug that were precipitated in the pore by removing the solvent.

### SUMMARY OF THE INVENTION

The invention comprises a method for forming small particles of a material, comprising:
a) providing a solid, or other material capable of having a pore structure, preferably pharmaceutically suitable matrix or carrier material having at least some small surface pores and/or internal pores, at least some of which communicate with the exterior surface of the matrix material, the pores being preferably smaller than about 200 nm;
b) providing a deposit material to be deposited in at least some of said pores;
c) providing a solvent, which may be a pure solvent or a mixture of components such as organic liquids, surfactants or supercritical fluids, that can dissolve the deposit material but does not significantly dissolve the matrix material under the conditions being used;
d) dissolving the deposit material in the solvent and putting the resulting solution in contact with the matrix material;
e) causing, or allowing, e.g., by capillary action, at least some of the solution of the deposit material to enter the pores of the matrix material;
f) removing sufficient, preferably all, solvent from the pores to cause the deposit material to be deposited, preferably in the form of discrete particles, in the pores of the matrix material.

More specifically, the invention comprises a method for introducing a water-insoluble, or poorly soluble, pharmaceutically active material into a living human or animal body, comprising:
a) providing a solid matrix material, or other material capable of having a pore structure, having at least some small internal pores, at least some of which communicate with the exterior surface of the matrix material;
b) providing a pharmaceutically active material to be deposited in at least some of said pores;
c) providing a solvent that can dissolve the pharmaceutically active material but does not significantly dissolve the matrix material under the conditions being used;
d) dissolving the pharmaceutically active material in the solvent and putting the resulting solution in contact with the matrix material;
e) causing at least some of the solution of the pharmaceutically active material to enter the pores of the matrix material;
f) removing sufficient solvent from the pores to cause particles of the pharmaceutically active material to deposit in the pores of the matrix material to provide a composition in which the pharmaceutically active material is at least partially in the form of particles contained in the pores of the matrix material;
g) introducing said composition into said living body;
h) allowing at least some of the particles of the pharmaceutically active material to enter from the pores of the matrix material into the blood stream or other physiological fluids or tissues of said living body, as by diffusion from the pores, or dissolution or partial dissolution of the matrix material, or both.

The invention also includes compositions made by the above process, as well as dispersions (including microemulsions, and the like) of such compositions.

In a preferred embodiment, the invention comprises the method described above wherein said solution of solid materials taken up by a porous matrix that is very soluble in water, such as lactose.

In another preferred embodiment, said solution of solid material is taken up by a porous matrix that is poorly soluble in water or is insoluble in water, such as silicone dioxide or sintered Teflon®.

In still another preferred embodiment, said solution of solid material is taken up in a porous matrix that is hygroscopic but not soluble in water, such as high molecular weight polyvinylpyrrolidone.

In yet another preferred embodiment, said solution of solid material is taken up in a matrix comprising nanometer scale tubes, such as carbon nanotubes.

In even another preferred embodiment, said solid material is dissolved in a supercritical fluid that may or may not contain surfactants, which is then taken up by the pores under high pressure. After removing the excess pressure, the supercritical fluid will be removed by evaporation.

It will be appreciated by those skilled in the art that surfactants may be used as necessary without departing from the spirit or scope of the invention. Similarly, methods such as melting or vaporizing of the material to be deposited in the matrix pores may be used as necessary without departing from the spirit or scope of the invention.

Very small particles of the prior art, e.g., nanoparticles, suffer from the disadvantage that they have a strong tendency to aggregate to their most stable form, i.e., a much larger particle size that has a smaller surface area per gram. It is a significant advantage of the instant invention that since the small particles are contained within the pores of a solid matrix, they can not migrate and/or aggregate; therefore, they are much more storage stable and can be readily shipped, milled, tabletized, pelletized, and otherwise manipulated.

The invention still further includes a composition comprising a solid, porous matrix having a preponderance of pores of an average size of about 200 nm or less, and containing within at least some of said pores particles of a solid material different from the material of the matrix, said particles of a solid material being at least partially soluble in a solvent that does not significantly dissolve said matrix.

### DETAILED DESCRIPTION OF THE INVENTION

The preparation of very small (e.g., less than ˜200 nanometers) particles is of great research and practical interest, particularly in the area of pharmaceuticals. In recent years, there has been a dramatic increase in the number of drugs discovered that have poor solubility in water. This is a problem because some degree of water solubility is necessary for a drug to be available to the human body as a whole (in other words, bioavailable). It is known that reducing the particle size of solid drugs or mixtures containing drugs can increase the solubility and bioavailability of such drugs, especially when the particle size is less than one micron in radius. During the last decade, research and development efforts have been put into ways to reduce the particle size of solids as much as possible. Today, particles as small as several hundred nanometers (known as nanoparticles) can be produced using established methods and equipment.

According to the present invention, it has been surprisingly found that it is possible to produce significantly smaller particles (˜25-50 nm or less) by precipitating materials, e.g., drugs, from solutions or suspensions within pores of solid matrixes, such as lactose and silicone dioxide, or other pore-containing matrixes, using a solvent or suspending agent that can dissolve or suspend the drug but not affect the matrix. For instance, the drug ibuprofen was deposited in lactose pores using ether as the solvent. It was found that the solubility of the ibuprofen in water increased by at least 16-fold.

This invention demonstrates that nanoparticles of drugs can be made simply and reliably, and the particle size is significantly smaller than nanoparticles currently being produced. Since reducing the particle size typically increases the drug solubility in water and enhances its bioavailability, it is reasonable to anticipate that particles produced using this method will provide significant improvement in drug delivery properties compared to nanoparticles produced by prior art methods.

For purposes of convenience, the present invention will sometimes hereinafter be described in terms of drugs, but it will be understood by those skilled in the art that numerous other materials can be substituted for the drug and numerous uses of the invention can be made other than to introduce drugs into a human or animal body.

The approach taken by the instant invention to making smaller particles of solid, poorly water-soluble or water-insoluble materials, preferably drugs, than possible using previous means is the use of a novel method, referred to as deposition by precipitation in pores, or DPP. The pores will be contained in, and/or on the surface of, a suitable matrix or carrier, which may be any solid or other material which contains accessible pores or in which such pores may be generated (e.g., a viscous liquid) and, in the case of drugs, is inert or otherwise biocompatible. The matrix may be water-soluble or, in the case of drugs, soluble in digestive and/or other bodily fluids, but this is not necessary. In addition, the matrix ideally will contain numerous small pores of, on average, less than about 200 mm, preferably less than about 100 nm, more preferably less than about 50 nm, most preferably about 25 nm or less, in size.

As used herein, the term “particle” is intended to include not only solid particles, but also any useful form of deposited material left behind in the pores after removal of solvent, such as droplets of solutions, microemulsions, micelles, suspensions, and the like. Accordingly, it will be understood that any given pore may be completely filled, as by a deposited liquid, and still be considered as containing a “particle” within the scope of the invention. As used herein, the term “matrix” is intended to include any suitable material, regardless of its configuration, which has suitable pores into which another material can be deposited by a method of this invention. As used herein, the term “pore” is intended to include any void space in a matrix that is larger than molecular scale, and thus can hold collections of molecules large enough to constitute a liquid, solid phase, particle; etc. As used herein, the phrase “communicate with the exterior surface” is intended to indicate that an accessible path for liquids or gases is available to or from an interior point to the matrix surface. As used herein, the term “dissolve” in intended to include not only dissolution in the usual physical chemical sense, but also the rendering of the material more accessible to, e.g., solvents, body fluids and tissues, and the like. As used herein, the term “suspension” is intended to include dispersions, emulsions, and the like, as indicated more fully below.

In a method of this invention, a drug is contained in a fluid which carries the drug into the pores of the chosen matrix, which may, for example, be an excipient useful in pharmaceuticals. While the drug-containing fluid may be either a liquid or a gas, preferably the drug may be dissolved in a solvent that does not dissolve the matrix under the process conditions chosen, and preferably does not dissolve the matrix at all. The fluid may be a solvent for the drug, but may also carry the drug as a suspension, an emulsion, microemulsion, micelles, colloidal suspension, and the like.

The matrix is preferably degassed, as by storing it in a vacuum, thus removing as much trapped air as desired from the pores, and then is placed in contact with the drug-bearing solution (or other drug-bearing fluid). The matrix is allowed to soak in the drug solution for a period of time sufficient to allow the solution to penetrate into pores of sufficiently small size (typically 100, preferably 25, nm or less). Penetration of the solution (or other fluid) into the pores of the matrix may be facilitated by the application of pressure or vacuum. After soaking for a sufficient time, most or all of the solvent is removed. Gross removal may be done by decanting the portion of solution not taken up into the pores. Further solvent removal may be accomplished by evaporation, including evaporation at a reduced pressure, and may be done at temperatures that are higher or lower than ambient, provided, of course, that the conditions chosen do not significantly harm the efficacy of the drug for its intended purpose. Removal of sufficient solvent or other fluid will cause precipitation of the drug in the pores of the matrix. Heating to remove solvent might result in formation of crystals of the drug on the surface of the matrix as well as in the pores. This may be minimized or avoided, if desired, by lyophilizing or reducing the temperature to produce precipitation in the pores before removing the solvent. Since there is typically a distribution of pore sizes, there will also be a distribution of particle sizes arising from this method. Typically, pore sizes of a matrix range from greater than 100 nm to as small as 1 mm, but this does not mean that all or any particles will be that small, since the particles can only be produced in regions accessible to the solution, i.e., are on the surface of the matrix and/or communicate with the surface of the matrix, and/or can be reached by adsorption or absorption. However, solutions of relatively low viscosity should readily penetrate into pores smaller than ˜20-25 nm, and the resulting precipitated particles should not exceed that size.

It will be understood from the above description that at least some of the pores of the matrix must be accessible to the drug-bearing solution or suspension.

The matrix may be soluble, slightly soluble or insoluble in water and/or body fluids or tissues, according to choice. If the matrix is soluble, the particles are dumped in a short time and then dissolve. If the matrix is insoluble, the drug will dissolve in the water or body fluid that penetrates into the pores, and then pass from the pores into the GI fluids. This will cause the drug to dissolve or release into the GI fluids more slowly, and may be the preferred mode for some drugs because it will create less of a degree of supersaturation. It will be apparent that release of the drug can be intentionally influenced by choice of matrix as well as choice of pore size, shape, concentration, location in/on the matrix, and the like. The loaded matrix may also be mixed or incorporated into other materials for purposes of manufacturing, tabletting, controlling drug dissolution (release rates, and the like). It will be apparent, therefore, that the rate of release and the place of release within the body can be controlled by judicious selection and handling of matrix material, granting flexibility of manufacturing, formulation and delivery well beyond anything taught in the prior art.

### DPP: Basic Principle

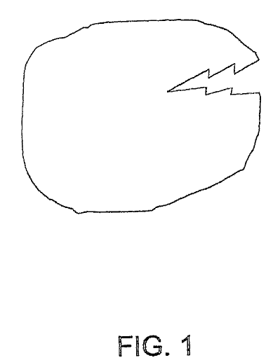

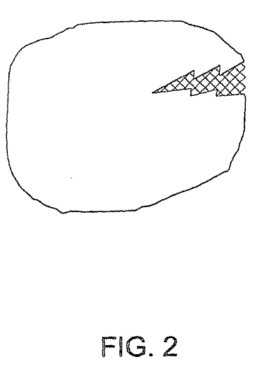

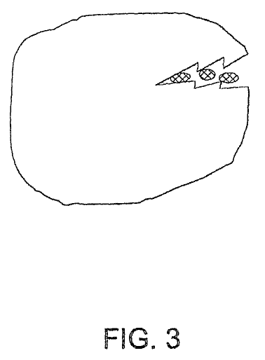

The formation of nanoparticles according to the invention is shown in schematic form in FIGS. 1 through 3, which illustrate, respectively, a solid matrix and its pores before deposition, after the solvent has been taken up, and after solvent removal, respectively. The key ideas are as follows.

1) Depositing a drug within a solid pore structure, the drug particles formed during precipitation will be in a confined space. As a result, these particles will not exceed the size of the pores which contain them. (It will be understood, of course, that for pores at the surface of the matrix, some solid drug may also form at the surface of the matrix.) For pharmaceutical solids, a typical desired pore size range for the pores particularly useful in this invention is ˜5-˜100 nm, but pores more especially desired for the present invention are in about the 20-50 nm range.

2) According to the well-known Kelvin Equation (P. Hiemenz and R. Rajagopalan, “Principles of Colloids and Surface Chemistry”, Marcel_Dekker, 1997, Chapter 6), the solubility of a drug increases with its curvature. For spherical particles, this is given by the equation

$$S = {S}_{0} exp ( \frac{2 γ {V}_{m}}{rRT} )$$

where S is the solubility of the drug in a liquid (e.g., water) in the presence of the drug particle of radius r, S₀ is the solubility of the bulk drug in the same liquid (assumes large particles, so r is large and the particle surfaces are approximated as being nearly flat), γ is the interfacial tension of the drug, Vₘ is the molar volume of the drug, R is the universal gas constant, and T is the absolute temperature (Kelvin). This equation is alternatively expressed as

$$RT ln \frac{S}{{S}_{0}} = \frac{2 M γ}{ρ r} \qquad$$

where M is the molar mass of the drug and ρ is the drug density.

3) Although the pores are perhaps better represented as deformed tubes or cylinders, as opposed to spheres, the ideas expressed in items 1) and 2) still hold. The relevant size parameter is related to the particle surface curvature, which for ideal cylinders would be related to the cylinder radius. In practice, these equations should not be taken too literally with respect to geometric shapes, since the pores are not ideal cylinders, but have irregular shapes and radii. In addition, it is not to be expected that the drug will form particles that are in the exact geometry of the pores; therefore, the particles are not likely to be ideal cylinders or other simple shapes with well-defined geometric parameters. However, the idea still holds that that small particles result in higher drug solubility, even if it is difficult to calculate the values exactly from a priori theory.

4) In addition to the drug trapped in the pores, some of the drug may also distribute onto the matrix outer surface, and may or may not form nanoparticles. However, the improved solubility results from the form of drug with the highest solubility. Therefore, in a mixture of large and small particles, the solubility will correspond to the solubility of the smallest particles. (However, the large particles may act as precipitation sites, which would make it more difficult to form supersaturated solutions on dissolution. The problem of precipitation from solutions is well known and exists for all methods that improve solubility and bioavailability by creating unstable, supersaturated solutions. This includes other nanoparticle technologies, and high-energy or amorphous solids, among others. Therefore, the precipitation problem should be considered as relevant to many solubility enhancement methods, and is not specific to DPP.)

### EXAMPLE 1

### DPP of Ibuprofen in Lactose Monohydrate

Precipitation of a drug with poor aqueous solubility into the pores of a matrix that is very soluble in water is preferred if it is desired to release all of the nanoparticles into the dissolution medium as quickly as possible. In this example, ibuprofen was used as the drug due to its low solubility in water, and lactose was used as the solid matrix. Ether was chosen as the carrier solvent because it readily dissolves ibuprofen, but does not dissolve lactose.

Since UV absorbance is directly proportional to ibuprofen content, UV absorption was used to assay ibuprofen dissolution rates. The following steps were performed:
a) Three grams of lactose monohydrate granules (which is known to be a porous material) were placed in a laboratory bench vacuum for 30 minutes to remove most of the air from the deeper pores in the granules.
b) 2 grams of ibuprofen were dissolved in 7 ml. ether.
c) 3 grams of the degassed lactose were mixed into the ibuprofen-ether solution. The resulting slurry was gently shaken for three hours at room temperature to allow the ibuprofen-ether solution to soak into the pores.
d) The ether was removed by evaporation at a low temperature (approximately −10° C.) under a vacuum (less than 0.1 atm pressure) for 3 days, leaving a dry solid, which was then pulverized in a mortar and pestle to make a fine powder. Since no decanting of the ether was done before evaporation, the dry solid contained 2 grams of ibuprofen and 3 grams of lactose
e) 0.6 gram of the ibuprofen-lactose granules, which contained ˜240 mg of ibuprofen (from part d), was placed in 50 ml. of a 0.01N HCl aqueous solution (pH 2) at 20° C.

The mixture was stirred using a magnetic stirring bar, and 1 ml. samples were taken after 5 and 15 minutes. Each sample was immediately filtered (0.2 micron filter), diluted 1:10 with more 0.01N HCl, and assayed by UV spectroscopy (258 nm). (Lactose does not show UV absorbance.) The absorbance of ibuprofen in the HCl solution (after 1:10 dilution) was 0.25 for the 5-minute sample and 0.19 for the 20-minute sample, corresponding to undiluted absorbance values of 2.5 and 1.9, respectively. For comparison, pure bulk ibuprofen powder (used as received from the vendor) was allowed to dissolve for 45 minutes in an identical receiving medium (0.01N HCl), and the absorbance was 0.15 for undiluted samples.

### EXAMPLE 2

### DPP of Ibuprofen in Beta-Lactose

Precipitation of a drug with poor aqueous solubility into the pores of a matrix that is very soluble in water is preferred if it is desired to release all of the nanoparticles into the dissolution medium as quickly as possible. In this example, ibuprofen was used as the drug due to its low solubility in water, and lactose was used as the solid matrix. Ether was chosen as the carrier solvent because it readily dissolves ibuprofen, but does not dissolve lactose.

Since small particles may have been present, samples were taken using microdialysis, since this is a method that collects filtered samples containing only dissolved drug molecules. The chemical assay for the dissolved concentration was done by HPLC. The following steps were performed:
a) Six grams of β-lactose granules (which is known to be a porous material) were placed in a vacuum oven for 30 minutes at less than 10 torr to remove most of the air from the deeper pores in the granules.
b) 11 grams of ibuprofen were dissolved in 50 ml. ether.
c) 6 grams of the degassed lactose were mixed into 15 ml. of the ibuprofen-ether solution. The resulting slurry was allowed to stand for one hour at room temperature to allow the ibuprofen-ether solution to soak into the pores.
d) As much excess solution as possible was carefully removed by decanting, and then using a glass dropper. The remaining ether was removed by evaporation at room temperature in a vacuum oven at less than 10 torr, for 2 hours. This left a dry solid, which was then pulverized in a mortar and pestle to make a fine powder.
e) The powder was weighed, and it was found that 6 grams of powder (containing lactose and ibuprofen) was recovered. Using an estimate of 20% porosity and true density of 1.5 for lactose, approximately 0.2-0.25 g of ibuprofen was deposited into the pores of 6 grams of lactose.
f) 1.5 grams of the ibuprofen-lactose powder, which contained ˜60 mg of ibuprofen (from part e) was placed in 50 ml. of a 0.01N HCl aqueous solution (pH ˜2) at 25° C.

The mixture was stirred using a magnetic stirring bar, and samples were taken every minute using microdialysis. Samples were immediately analyzed by HPLC.
g) For comparison, the same procedure was done using pure ibuprofen powder, mixing 100 mg into 50 ml. of 0.01N HCl. Samples were taken every minute for 10 minutes.

h) For dissolution of the pure ibuprofen, approximately 350 mcg had dissolved after two minutes, and approximately 790 mcg had dissolved after 5 minutes. For the ibuprofen-lactose formulations, approximately 600 mcg of ibuprofen had dissolved in the first two minutes, and 4500 mcg had dissolved after five minutes. (For comparison, the solubility of pure ibuprofen at pH 2 is approximately 80 mcg/ml., which corresponds to 4000 mcg dissolved in 50 ml.)

### EXAMPLE 3

### DPP of Ibuprofen in Beta-Lactose

Precipitation of a drug with poor aqueous solubility into the pores of a matrix that is very soluble in water is preferred if it is desired to release all of the nanoparticles into the dissolution medium as quickly as possible. In this example, ibuprofen was used as the drug due to its low solubility in water, and lactose was used as the solid matrix. Ether was chosen as the carrier solvent because it readily dissolves ibuprofen, but does not dissolve lactose.

Since small particles may have been present, samples were taken using microdialysis, since this is a method that collects filtered samples containing only dissolved drug molecules. The chemical assay for the dissolved concentration was done by HPLC. The following steps were performed:
a) Six grams of microcrystalline cellulose (MCC) placed in a vacuum oven for 30 minutes at less than 10 torr to remove most of the air from the deeper pores in the granules.
b) 11 grams of ibuprofen were dissolved in 50 ml. ether.
c) 6 grams of the degassed MCC were mixed into 15 ml. of the ibuprofen-ether solution. The resulting slurry was allowed to stand for one hour at room temperature to allow the ibuprofen-ether solution to soak into the pores. (Ether does not dissolve MCC.)
d) As much excess solution as possible was carefully removed by decanting, and then using a glass dropper. The remaining ether was removed by evaporation at room temperature in a vacuum oven at less than 10 torr, for 2 hours. This left a dry solid, which was then pulverized in a mortar and pestle to make a fine powder.
e) The powder was weighed, and it was found that 6 grams of powder (containing MCC and ibuprofen) was recovered. Using an estimate of 20% porosity and true density of 1.5 for lactose, approximately 0.2-0.25 g of ibuprofen was deposited into the pores of 6 grams of MCC.
f) 1.5 grams of the ibuprofen-MCC powder, which contained ˜60 mg of ibuprofen (from part e) was placed in 50 ml. of a 0.01 N HCl aqueous solution (pH 2) at 25° C. The mixture was stirred using a magnetic stirring bar, and samples were taken every minute using microdialysis. Samples were immediately analyzed by HPLC.
g) For comparison, the same procedure was done using pure ibuprofen powder, mixing 100 mg into 50 ml. of 0.01N HCl. Samples were taken every minute for 10 minutes.

h) For dissolution of the pure ibuprofen, approximately 190 mcg had dissolved after one minute, approximately 350 mcg had dissolved after two minutes, and approximately 790 mcg had dissolved after 5 minutes. For the ibuprofen-MCC, approximately 1200 mcg had dissolved after 1 minute. The amount dissolved increased slowly for the next several minutes, with approximately 2000 mcg dissolved after five minutes. After 10 minutes, however, approximately 4500 mcg had dissolved.

### RESULTS AND DISCUSSION

From the data given in Example 1, it can be seen that the dissolution of ibuprofen from the nanoparticle form produced by this invention is much faster than what was measured using bulk ibuprofen. Approximately 16 times more ibuprofen was in solution after 5 minutes from the nanoparticle form than was in solution after 45 minutes from the bulk drug. This indicates that the solubility of the ibuprofen was also increased as a result of the nanoparticle formation.

Ibuprofen is a weak acid, with a pKa of ápproximately 4.5, and it is more than 99% unionized at a pH of 2. This is important because the unionized form of ibuprofen is poorly soluble in aqueous solutions, unless complexed with other agents. However, in this acidic solution of 0.01 N HCl, no agents were present that could complex or otherwise alter the drug solubility. Thus, factors such as salt formation, complexation, etc., can be ruled out as means for increasing the ibuprofen dissolution rate and solubility. This clearly suggests that the increase is due to a significant particle size reduction of the ibuprofen (as compared to the bulk drug).

There is one well-known source of experimental error in this experiment, but it does not invalidate the conclusions that can be drawn from the experiment. If nanoparticles are formed, many will pass through a 0.2 micron filter (see Step e) above). Thus, although the amount of ibuprofen that was dissolved when the UV assay was done is known, it is not known exactly how much ibuprofen was dissolved from the DPP particles before filtering and dilution. Two comments are appropriate here. First, if particles did pass through the filter and dissolved (fully or partially) after dilution, this supports the notion that nanoparticles were indeed formed by the DPP method. Second, the UV assay was done less than five minutes after taking the sample (including filtering and dilution). Thus, even if particles were present that continued to dissolve after filtering, the dissolution rate would still be much higher than what was seen for the bulk ibuprofen.

From the above, it can be stated with reasonable confidence that DPP does lead to the production of nanoparticles, which leads, in turn, to faster dissolution of ibuprofen. In addition, there was nothing in this experiment that would be considered specific to ibuprofen (except for using a low pH aqueous receiver medium). Thus, it is expected that DPP would work for other drugs as well, in the sense of forming small particles and increasing dissolution rates.

Example 2 did not show an increase in solubility of the drug at long times, but did demonstrate an increased dissolution rate at early times (first five minutes or more) compared to the pure ibuprofen dissolution. For instance, after 1 minute, nearly twice the amount of ibuprofen had dissolved from the lactose formulation compared to the pure ibuprofen powder. After five minutes, the amount of ibuprofen dissolved was close to the nominal solubility (4500 mcg in 50 ml., or 90 mcg/ml., which is close to the solubility of 80 mcg/ml. at pH 2). Similar results were seen from Example 3, which also illustrated a dramatic increase in early time dissolution rates. At longer times, the total amount dissolved did not dramatically increase, so an increased solubility was not explicitly demonstrated. (This effect is probably related to the presence of some larger particles on the surface of the matrix, which served as nucleation or precipitation sites. This is a known effect that can likely be minimized or eliminated with further formulation development using the DPP technology.)

Even though an increased solubility was not explicitly demonstrated, the increased early dissolution rates imply that either small particles or increased solubility did occur. This can be seen by interpreting the data using modifications of the Noyes-Whitney or Hixson-Crowell models (P. Sinko, “Martin's Physical Pharmacy and Pharmaceutical Sciences, 5^{th} Edition”, Lippincott, Williams and Wilkins, 2006, Chapter 13.) Both of these dissolution models predict that the dissolution rate of a drug increases with increasing drug solubility and/or increasing surface area. Since the pure ibuprofen was in the form of a fine powder, the increased dissolution rates from the DPP formulations implies either an increased solubility or smaller particle size (reflected in higher surface area per gram of dissolving material).

What is claimed is:

1. A method for forming particles of a material, comprising:
a) providing a matrix material having at least some pores at least some of which communicate with the exterior surface of the matrix material;
b) providing a deposit material to be deposited in at least some of said pores;
c) providing a fluid that can dissolve or suspend the deposit material but does not significantly dissolve the matrix material under the conditions being used;
d) dissolving or suspending the deposit material in the fluid and putting the resulting solution or suspension in contact with the matrix material;
e) allowing at least some of the solution or suspension of the deposit material to enter the pores of the matrix material;
f) removing sufficient fluid from the pores to cause the deposit material to be deposited in the pores of the matrix material.

2. A method of claim 1 in which the average pore size of the matrix material is less than about 200 nm.

3. A method of claim 1 in which the matrix material is at least partially water-soluble.

4. A method of claim 1 in which the matrix material is essentially insoluble in water.

5. A method of claim 1 in which the deposit material is a pharmaceutically active material.

6. A composition made by a method of claim 1.

7. A method for forming particles of a water-insoluble or poorly water-soluble, pharmaceutically active material, comprising:
a) providing a matrix material having at least some pores, at least some of which communicate with the exterior surface of the matrix material;
b) providing a pharmaceutically active material to be deposited in at least some of said pores;
c) providing a fluid that can dissolve or suspend the pharmaceutically active material but does not significantly dissolve the matrix material under the conditions being used in step (e);
d) dissolving or suspending the pharmaceutically active material in the fluid and putting the resulting solution or suspension in contact with the matrix material;
e) allowing at least some of the solution or suspension of the pharmaceutically active material to enter the pores of the matrix material;
f) removing sufficient fluid from the pores to cause particles of the pharmaceutically active material to deposit in the pores of the matrix material to provide a composition in which the pharmaceutically active material is at least partially in the form of discrete particles contained in the pores of the matrix material.

8. A composition comprising a solid, porous matrix having a preponderance of pores of an average size of about 200 nm or less, and containing within at least some of said pores particles of a solid material different from the material of the matrix, said particles of a solid material being at least partially soluble in a solvent that does not significantly dissolve said matrix.

9. A method of introducing a water-insoluble or poorly water-soluble material into a living human or animal body comprising introducing into said body a composition of claim 6 in which said water-insoluble or poorly water-soluble material is contained in the pores of the matrix material.

10. A suspension of a composition of claim 6.

11. A method of claim 7 in which the average pore size of the matrix material is less than about 200 nm.

12. A method of claim 7 in which the matrix material is at least partially water-soluble.

13. A method of claim 7 in which the matrix material is essentially insoluble in water.

14. A composition made by a method of claim 7.

15. A method of introducing a water-insoluble or poorly water-soluble material into a living human or animal body comprising introducing into said body a composition of claim 7 in which said water-insoluble or poorly water-soluble material is contained in the pores of the matrix material.

16. A method of claim 1 in which the matrix material is in the form of a tape.

17. A method of claim 7 in which the matrix material is in the form of a tape.

18. A method of claim 12 in which the matrix material is water soluble.

19. A suspension of a composition of claim 14.
